(19) United States
(12) Patent Application Publication
(10) Pub. No.: US 2025/0186268 A1
(43) Pub. Date: Jun. 12, 2025

(54) DETECTON UNIT WITH CONDUCTIVE RUBBER

(71) Applicant: Raja Singh Tuli, Montreal (CA)

(72) Inventor: Raja Singh Tuli, Montreal (CA)

(21) Appl. No.: 18/953,570

(22) Filed: Nov. 20, 2024

Related U.S. Application Data
(60) Provisional application No. 63/606,791, filed on Dec. 6, 2023.

(51) Int. Cl. A61F 13/42; G01N 27/22

(52) U.S. Cl. CPC A61F 13/42; G01N 27/225; A61F 2013/424

(57) ABSTRACT

Conductive rubber is provided on a detection device configured to be releasably attached to an absorbent article and to operate in combination with the spaced-apart conductive lines in the absorbent article for wetness/saturation detection and estimation of the absorbent article, in order to facilitate the coupling/connection between the detection device and the conductive lines in an absorbent article so as to improve the accuracy of the wetness and/or saturation detection in the absorbent article.

### CROSS-REFERENCE TO RELATED APPLICATIONS

This application claims priority to U.S. provisional patent application No. 63/606,791, entitled “DETECTON UNIT WITH CONDUCTIVE RUBBER,” filed on Dec. 6, 2023. The content of this U.S. provisional patent application is hereby incorporated by reference in its entirety for all purposes.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not Applicable

### STATEMENT REGARDING PRIOR DISCLOSURES BY THE INVENTOR OR A JOINT INVENTOR

Not Applicable

### BACKGROUND OF THE INVENTION

### Field of the Invention

The present disclosure relates to wetness and/or saturation detection devices for an absorbent article, in particular to wetness and/or saturation detection devices with conductive rubber.

### Description of Related Art

Disposable absorbent article such as disposable diaper is a product that is capable of receiving and retaining bodily exudates or excretions so as to prevent contamination of the clothing or external environment. As an example, with a disposable diaper, the user is allowed to urinate or defecate without the use of a toilet. In addition to diapers, there are numerous other types of disposable absorbent articles such as e.g. under pads, incontinence pads, fitted briefs, belted shields, liners, all-in-one pads, pull-up incontinence pants, training pants, protective underwear, catamenial napkins, and incontinence guards etc. It is to be understood that the list of disposable absorbent articles identified above is not exhaustive and that these and other absorbent articles can be used with the present disclosure and are within the scope of the present disclosure. It is also to be understood that a reference in this specification to any one such article, such as a “diaper” is to be taken to be a reference to any and all other suitable absorbent articles including incontinence garments, pads and the like.

In order to prevent contamination of the clothing or external environment, disposable absorbent article is provided with an absorbent core capable of receiving and retaining bodily exudates or excretions, and a substantially liquid impervious layer. In general, disposable absorbent products consist of a layered construction, which allows the bodily exudates or excretions to be distributed and transferred to the absorbent core where they are retained in. In everyday use, a disposable absorbent article may be used until the absorbent core is saturated with e.g. bodily exudates or excretions. When the absorbent core is saturated, the disposable absorbent article needs to be removed, disposed of, and replaced with a clean and dry article.

Without a solution for wetness and/or saturation detection and estimation of an absorbent article, the user wearing the absorbent article might be left in their own urine and feces for extended periods of time, causing many health problems. For many years, a variety of designs have been developed for detecting and signaling the wetness and/or saturation in an absorbent article such as a diaper. However, the accuracy of the wetness and/or saturation detection is not satisfactory and/or remains doubtful. Therefore, there is a need to improve the accuracy of the wetness and/or saturation detection.

Furthermore, in the prior art, there is no disclosure that adopts conductive rubber to facilitate the coupling/connection between the detection device and the sense electrodes so as to improve the accuracy of the wetness and/or saturation detection in an absorbent article. In addition to conductive rubber, there are numerous other types of conductive material that can be used with the present disclosure and are within the scope of the present disclosure, such as e.g. conductive foam, conductive pliable material, conductive material that is soft, etc. It is to be understood that a reference in this specification to any one such material, such as a “rubber” is to be taken to be a reference to any and all other suitable material such as foam, pliable material, and the like.

### BRIEF SUMMARY OF THE INVENTION

Many care facilities have no efficient way to determine, monitor, and schedule service and visits based on the real time needs of the patient. Patients are often left in their own urine and feces for extended periods of time, which may cause health problems. This leads to an increased demand for alternative incontinence management solutions. In order for an accurate wetness and/or saturation detection in an absorbent article, a reliable and good cooupling/connection between a detection device and the sense electrodes is critical. In accordance with the present subject matter, a wetness/saturation detection device is provided with conductive rubber, in order to facilitate the coupling/connection between the detection device and the sense electrodes thereby improving the accuracy of the wetness and/or saturation detection in an absorbent article.

Embodiments are presented herein of, inter alia, a wetness/saturation detection device for an absorbent article.

According to an embodiment of the present disclosure, a detection device is provided that is configured to be releasably attached to an absorbent article. The detection device comprises: a first half provided with at least one strip of flexible conductive material; and a second half. And the absorbent article is provided with at least two conductive lines under its porous, liquid pervious layer. When the detection device is attached to the absorbent article, its first and second halves are pinched towards each other under pressure, with the absorbent article being provided therebetween, and the at least one strip of flexible conductive material under such pressure is pushed against the absorbent article and contacts the respective conductive lines through the porous, liquid pervious layer of the absorbent article.

According to another embodiment of the present disclosure, a detection device is provided that is configured to be releasably attached to an absorbent article. The detection device comprises: a first half provided with at least two conducts; and a second half provided with at least one strip of flexible material. And the absorbent article is provided with at least two conductive lines under its porous, liquid pervious layer. When the detection device is attached to the absorbent article, its first and second halves are pinched towards each other under pressure, with the absorbent article being provided therebetween, and the at least one strip of flexible material under such pressure pushes the absorbent article against the first half, thereby making the at least two conductive lines in the absorbent article contact the respective contacts on the first half through the porous, liquid pervious layer of the absorbent article.

According to an embodiment of the present disclosure, a detection device is provided that is configured to be releasably attached to an absorbent article. The detection device comprises: a first half, and a second half. And at least one strip of flexible material is provided on the inner side of one of the first and second halves, while at least one ridge is provided on the inner side of the other one of the first and second halves along the length direction of the detection device.

This summary is intended to provide a brief overview of some of the subject matter described in this document. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF THE DRAWINGS

The various preferred embodiments of the present invention described herein can be better understood by those skilled in the art when the following detailed description is read with reference to the accompanying drawings. The components in the figures are not necessarily drawn to scale and any reference numeral identifying an element in one drawing will represent the same element throughout the drawings. The figures of the drawing are briefly described as follows.

FIG. 1 illustrates an exemplary absorbent article in an exploded perspective view according to an embodiment of the present disclosure;

FIG. 2 illustrates an exemplary disposable absorbent article with two spaced-apart conductive lines being provided on the top side of its substantially liquid impervious layer according to an embodiment of the present disclosure;

FIG. 3 illustrates an exemplary detection device (e.g. pod) that is configured to be releasably attached to an absorbent article and to operate in combination with the spaced-apart conductive lines in the absorbent article for wetness/saturation detection and estimation of the absorbent article, according to an embodiment of the present disclosure; and

FIGS. 4A and 4B schematically illustrate an exaggerated sectional view of a detection device with conductive rubber or with rubber respectively when being clipped on an absorbent article, according to an embodiment of the present disclosure;

FIG. 4C schematically illustrates an exaggerated sectional view of a detection device as illustrated in FIG. 4A in open configuration, according to an embodiment of the present disclosure; and

FIGS. 5A and 5B schematically illustrates an exaggerated sectional and front views of a detection device respectively, according to an embodiment in its open configuration.

While the features described herein are susceptible to various modifications and alternative forms, specific embodiments thereof are shown by way of example in the drawings and are herein described in detail. It should be understood, however, that the drawings and detailed description thereto are not intended to be limiting to the particular form disclosed, but on the contrary, the intention is to cover all modifications, equivalents and alternatives falling within the spirit and scope of the subject matter as defined by the appended claims.

### DETAILED DESCRIPTION OF THE INVENTION

Disposable absorbent article such as disposable diaper is a product that is capable of receiving and retaining bodily exudates or excretions so as to prevent contamination of the clothing or external environment. As an example, with a disposable diaper, the user is allowed to urinate or defecate without the use of a toilet. In addition to diapers, there are numerous other types of disposable absorbent articles such as e.g. under pads, incontinence pads, fitted briefs, belted shields, liners, all-in-one pads, pull-up incontinence pants, training pants, protective underwear, catamenial napkins, and incontinence guards etc. It is to be understood that the list of disposable absorbent articles identified above is not exhaustive and that these and other absorbent articles can be used with the present disclosure and are within the scope of the present disclosure. It is also to be understood that a reference in this specification to any one such article, such as a “diaper” is to be taken to be a reference to any and all other suitable absorbent articles including incontinence garments, pads and the like.

In order to prevent contamination of the clothing or external environment, disposable absorbent article is provided with an absorbent core capable of receiving and retaining bodily exudates or excretions, and a substantially liquid impervious layer. In general, disposable absorbent products consist of a layered construction, which allows the bodily exudates or excretions to be distributed and transferred to the absorbent core where they are retained in. In everyday use, a disposable absorbent article may be used until the absorbent core is saturated with e.g. bodily exudates or excretions. When the absorbent core is saturated, the disposable absorbent article needs to be removed, disposed of, and replaced with a clean and dry article.

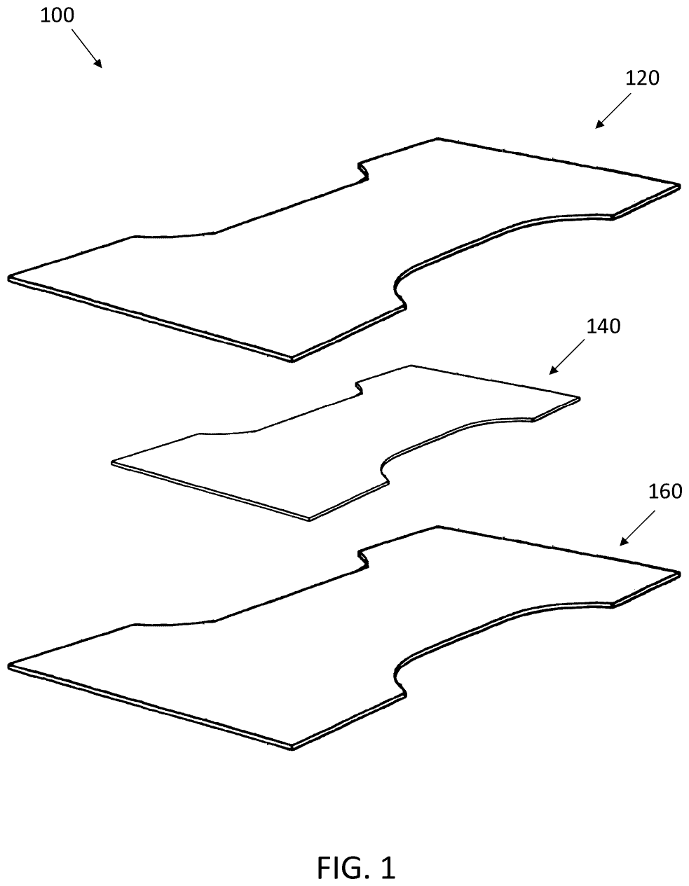

FIG. 1 illustrates an exemplary disposable absorbent article in an exploded perspective view. As illustrated, a disposable absorbent article 100 primarily consists of an absorbent core 140 sandwiched between a liquid pervious layer 120 and a substantially liquid impervious layer 160.

As illustrated in the exemplary diaper of FIG. 1, a disposable absorbent article 100 has a substantially liquid impervious layer 160 configured to prevent the bodily exudates or excretions absorbed and retained in the absorbent core 140 from wetting articles, such as bed sheets and undergarments, which contact the disposable absorbent article 100. On top of the layer 160 is disposed an absorbent core 140 made of a superabsorbent material. On top of the absorbent core 140 is a liquid pervious layer 120 that is joined to the layer 160 in an assembled state of the disposable absorbent article and is placed next to the skin of the user when in use. Additional structural features such as additional layer(s), elastic members and fastening means for securing the article in place, such as tape tab fasteners, may also be included.

The liquid pervious layer 120 is configured to be penetrable by bodily exudates and excretions in a direction into the absorbent core 140 to enable them to be absorbed and retained in the underlying absorbent core 140. It is appreciated that the layer 120 may be made of a variety of liquid pervious materials, e.g. nonwoven fabric. It is also appreciated that the material of the liquid pervious layer 120 is in general made of fibers bonded together with holes or apertures being formed through the material.

The absorbent core 140 is made up of hydrophilic superabsorbent polymers (SAP) and fibrous material, as a non-limiting example. The polymers act like tiny sponges that retain many times their weight in liquid.

The substantially liquid impervious layer 160 is made of a material substantially impervious to liquids. As an example, the substantially liquid impervious layer 160 may be manufactured from a thin plastic film, although other liquid impervious materials may also be used. As described above, the substantially liquid impervious layer 160 is configured to prevent the bodily exudates or excretions absorbed and retained in the absorbent core from wetting articles, such as bed sheets and undergarments, which contact the diaper.

As illustrated in the exemplary diaper of FIG. 1, the layers 120 and 160 are coextensive and have generally larger dimension, in length and/or width, than the absorbent core 140.

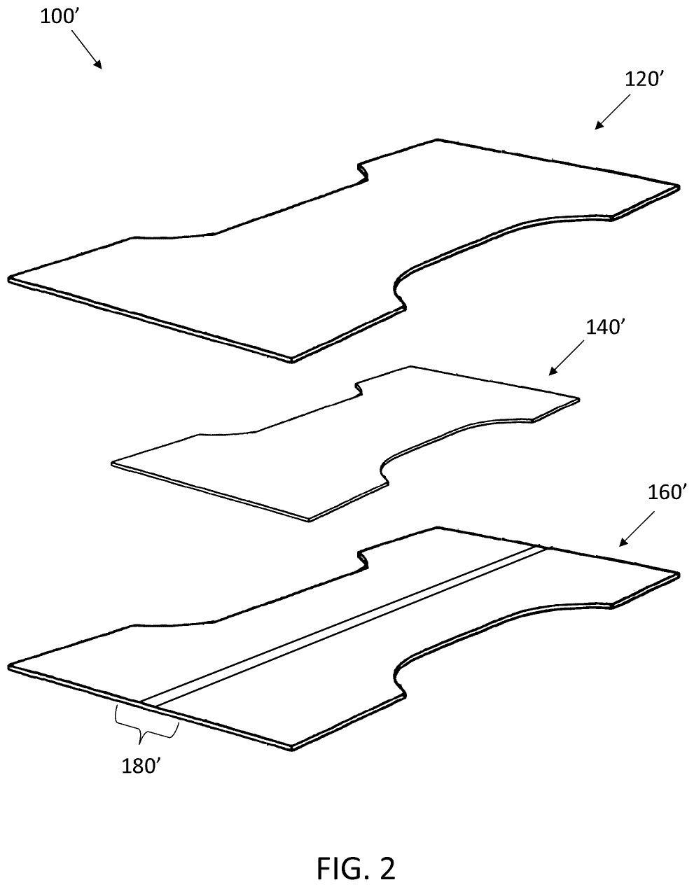

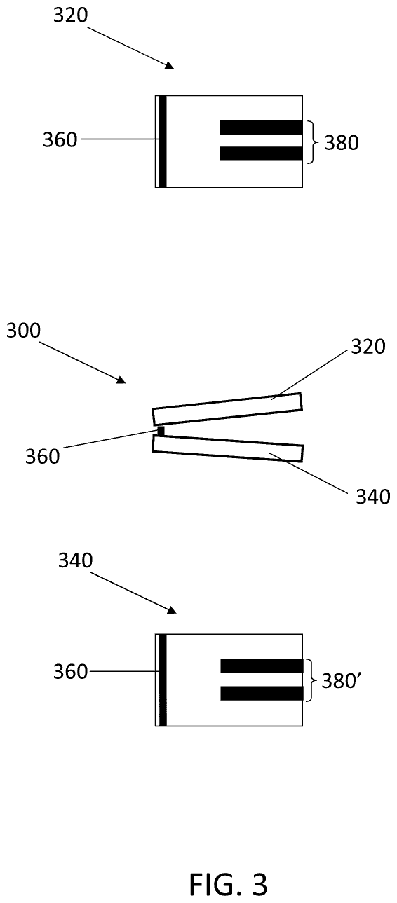

In order for wetness/saturation detection and estimation of an absorbent article, in particular to detect the presence and/or amount and/or saturation of the bodily exudates or excretions in the absorbent article, in particular in its absorbent core, a number of (e.g. at least two) spaced-apart conductive lines are provided as electrodes on the top side (i.e. the side facing the absorbent core) of the substantially liquid impervious layer along the length of the absorbent article, in an embodiment of the present disclosure. In FIG. 2, an exemplary disposable absorbent article 100′ is depicted with two spaced-apart conductive lines 180′ being provided on the top side of the substantially liquid impervious layer 160′, as an example. The spaced-apart conductive lines 180′ in the disposable absorbent article 100′ operate in cooperation with a detection device (e.g. pod) 300 (to be described below in reference to FIG. 3), for wetness/saturation detection and estimation of the absorbent article 100′.

In order for wetness/saturation detection and estimation of an absorbent article, a detection device (e.g. pod) is configurable to be attached (preferably, releasably attached) to the absorbent article and to operate in combination with the spaced-apart conductive lines in the disposable absorbent. According to an embodiment of the present disclosure and e.g. as illustrated in FIG. 3, an exemplary detection device 300 (e.g. pod) primarily consists of two halves 320 and 340 that are pivotably coupled to each other with a pivotal connection 360. In according to an embodiment of the present disclosure, the first half 320, which contacts the liquid pervious layer of an absorbent article when the detection device 300 is attached to (e.g. clipped on) the absorbent article, is provided with a number of (e.g. at least two) conductive rubber strips 380 on its inner side (i.e. the side facing the other half, i.e. the second half). Two conductive rubber strips 380 are schematically illustrated in FIG. 3, as a non-limiting example.

In operation, the detection device 300 as illustrated in FIG. 3 is clipped on a disposable absorbent article (e.g. 100′ as illustrated in FIG. 2) at one of its waist end edges, with the conductive rubber strips 380 on the detection device 300 being aligned with the conductive lines (e.g. 180′ in FIG. 2). When being clipped on the absorbent article, the two halves 320 and 340 of the detection device 300 are configured to be pinched towards each other under pressure in order to secure the detection device 300 on the absorbent article. Because of its flexibility and conductivity, the conductive rubber strips 380 under such pressure are pushed against the liquid pervious layer 120′ and thus some portions of them are forced to bend and pass through the holes in the liquid pervious layer 120′ and thus directly contact the respective conductive lines 180′ provided on the substantially liquid impervious layer 160′, thereby providing a direct connection or contact between the conductive lines and the detection device.

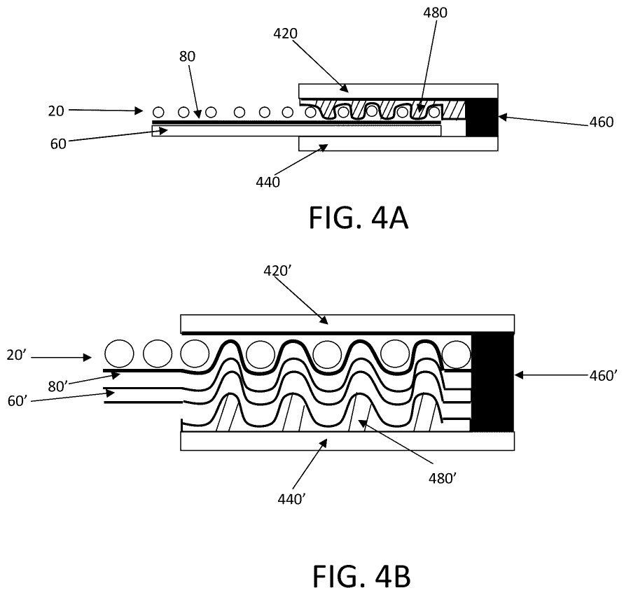

FIG. 4A schematically illustrates an exaggerated sectional view of a detection device clipping on an absorbent article, wherein the detection device is provided with conductive rubber (e.g. a number of conductive rubber strips). As illustrated in FIG. 4A, a detection device is primarily composed of a first half 420 and a second half 440 that are pivotably coupled to each other with a pivotal connection 460. The detection device is also provided with a conductive rubber strip 480 (shown shaded) on the inner side of the first half 420. In FIG. 4A the detection device is illustrated as being clipped on an absorbent article, for which only a liquid pervious layer 20, a substantially liquid impervious layer 60, and a conductive line 80 is illustrated for reasons of simplification. The liquid pervious layer 20 is illustrated by a series of circles (which represents its fibers) to demonstrate its construction with holes. As illustrated in FIG. 4A, when the detection device is clipped on the absorbent article, the clamping force between the two halves 420 and 440 forces the conductive rubber strip 480 to pass through the holes in the liquid pervious layer 20 and thus directly contact the conductive line 80 on the substantially liquid impervious layer 60, thereby providing a direct connection or contact between the conductive lines and the detection device.

In addition or alternatively, in a detection device 300 a number of (e.g. at least two) conductive rubber strips 380′ may be provided on the second half, which contacts the substantially liquid impervious layer of an absorbent article when the detection device 300 is attached to (e.g. clipped on) the absorbent article, on its inner side (i.e. the side facing the other half), according to an embodiment of the present disclosure. Two rubber strips 380′ are schematically illustrated in FIG. 3, as a non-limiting example.

In operation, the detection device 300 as illustrated in FIG. 3 is clipped on a disposable absorbent article (e.g. 100′ as illustrated in FIG. 2) at one of its waist end edges, with the rubber strips 380′ on the detection device 300 being aligned with the conductive lines (e.g. 180′ in FIG. 2). When being clipped on an absorbent article, the two halves 320 and 340 of the detection device 300 are configured to be pinched towards each other under pressure in order to secure the detection device 300 on the absorbent article. Because of its flexibility, the rubber strips 380′ under such pressure are pushed against the substantially liquid impervious layer 120′ which forces some portions of the substantially liquid impervious layer 120′ to bend and in turn forces the conductive lines (e.g. 180′ in FIG. 2) to bend and pass through the holes in the liquid pervious layer 120′ and thus directly contact the contacts on the first half 320, thereby providing a direct connection/contact between the conductive lines and the detection device. It is to be noted that in the embodiment with rubber strips being provided on the half that contacts the substantially liquid impervious layer may be incorporated with the embodiment with conductive rubber strips on the other half of the detection device, that is, the contacts on the first half 320 of the detection device 300 may be implemented as the conductive rubber strips 380. Alternatively, the contacts on the first half 320 may be implemented in other appropriate manners, e.g. implemented as a number of conductive lines or pads provided on the inner side of the first half 320.

FIG. 4B schematically illustrates an exaggerated sectional view of a detection device clipping on an absorbent article, wherein the detection device is provided with rubber. As illustrated in FIG. 4B, a detection device is primarily composed of a first half 420′ and a second half 440′ that are pivotably coupled to each other with a pivotal connection 460′. The detection device is also provided with a rubber strip 480′ (shown shaded) on the inner side of the second half 440′. In FIG. 4B the detection device is illustrated as being clipped on an absorbent article, for which only a liquid pervious layer 20′, a substantially liquid impervious layer 60′, and a conductive line 80′ is illustrated for reasons of simplification. The liquid pervious layer 20′ is illustrated by a series of circles (which represents its fibers) to demonstrate its construction with holes. As illustrated in FIG. 4B, when the detection device is clipped on the absorbent article, the clamping force between the two halves 420 and 440 pushes the rubber strip 480′ against the first half 420′, which in turn forces the substantially liquid impervious layer 60′ and the conductive line 80′ to pass through the holes in the liquid pervious layer 20′ and directly contact the contacts provided on the inner side of the first half 420′, thereby providing a direct connection or contact between the conductive lines and the detection device.

FIG. 4C schematically illustrates an exaggerated sectional view of a detection device as illustrated in FIG. 4A in an open configuration, i.e. when its two halves are not pinched towards each other. As illustrated in FIG. 4C, a detection device is primarily composed of a first half 420 and a second half 440 that are pivotably coupled to each other with a pivotal connection 460. The detection device is also provided with a conductive rubber strip 480 (shown shaded) on the inner side of the first half 420. In FIG. 4C the detection device is illustrated as being in open configuration, with an absorbent article being placed between its two halves 420 and 440, for which only a liquid pervious layer 20, a substantially liquid impervious layer 60, and a conductive line 80 is illustrated for reasons of simplification. Similar as in FIG. 4A, the liquid pervious layer 20 is illustrated by a series of circles (which represents its fibers) to demonstrate its construction with holes. In FIG. 4C, since the two halves 420 and 440 are in an open configuration, i.e. are not pinched towards to each other, the conductive rubber strip 480 on the first half 420 contacts neither the second half 440, nor the absorbent article placed between the two halves, and therefore the conductive rubber strip 480 is in its intact state, i.e. it has no deformation due to the clamping force between the two halves 420 and 440.

Similarly, the conductive rubber strip 480′ as illustrated in FIG. 4B also has no deformation in its intact state, i.e. when it is in an open configuration, because the two halves 420′ and 440′ are not pinched towards to each other.

It is appreciated that the number of rubber strips (either conductive rubber strips e.g. 380 in FIG. 3, or rubber strips e.g. 380′ in FIG. 3) may be replaced by a respective single (conductive) rubber strip that overlays all the conductive lines provided on the liquid pervious layer of an absorbent article when the detection device is attached to the absorbent article. When a single conductive rubber strip is provided, the conductive rubber strip is configured to be conductive only in the direction normal to the inner side of the detection device's first half and in the direction parallel to the conductive lines in the plane of the inner side of the detection device's first half, that is, the conductive rubber strip is not conductive in the direction vertical to the conductive lines in the plane of the inner side of the detection device's first half, in order to avoid the potential short circuit between these conductive lines.

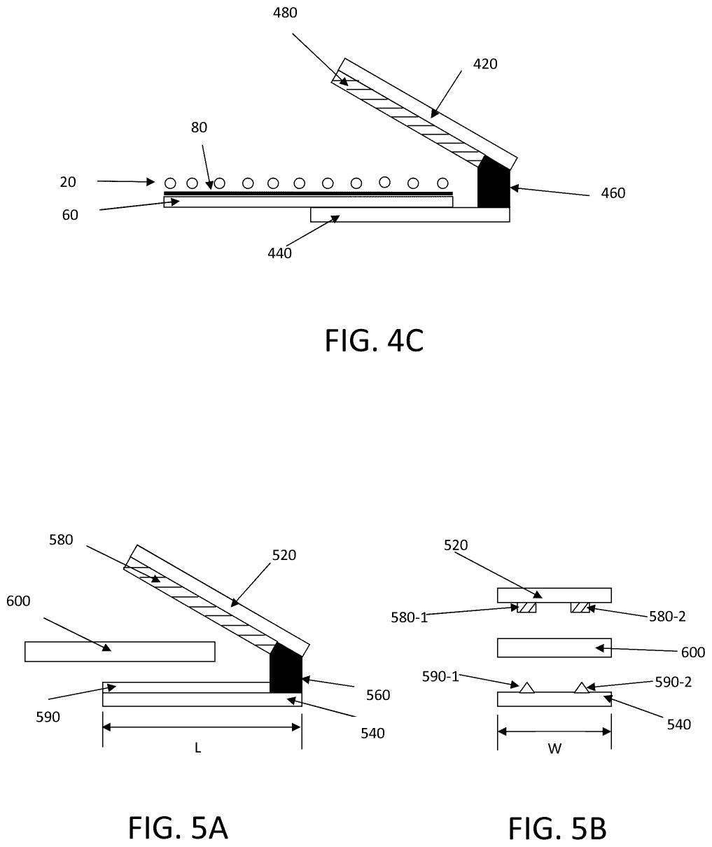

In an embodiment of the present disclosure, (conductive) rubber strip(s) is provided on the inner side of one half of a detection device (e.g. pod), while ridge(s) is provided on the inner side of the other half of the detection device (e.g. pod) in order to facilitate pushing the absorbent article against the inner side of the one half, in particular against the (conductive) rubber strip(s) provided on the inner side of the one half. FIGS. 5A and 5B schematically illustrates an exaggerated sectional and front views of a detection device according to an embodiment in its open configuration, respectively. As illustrated in FIG. 5A, a detection device is primarily composed of a first half 520 and a second half 540 that are pivotably coupled to each other with a pivotal connection 560. The detection device is also provided with a (conductive) rubber strip 580 (shown shaded) on the inner side of the first half 520. In FIG. 5A the detection device is illustrated in an open configuration, with an absorbent article 600 being provided between the two halves 520 and 540. Further, a ridge 590 is shown in FIG. 5A as being provided on the inner side of the second half 540 along the length direction L of the detection device. As can be seen from FIG. 5B, two (conductive) rubber strip 590-1 and 590-2 are provided on the inner side of the first half 520 along the length direction L of the detection device (i.e. orthogonal to the width direction W of the detection device), while two corresponding ridges 590-1 and 590-2 are provided on the inner side of the second half 540, opposite to the two (conductive) rubber strip 590-1 and 590-2, thereby facilitate pushing the absorbent article 600 against the inner side of the first half 520, in particular against the (conductive) rubber strips 580-1 and 580-2. It is to be noted that the configuration as illustrated in FIGS. 5A and 5B is for illustration only, and other configurations with ridge(s) are envisaged and fall within the scope of the present disclosure. As an example, (conductive) rubber strip(s) may be provided on the second half, while ridge(s) may be provided on the first half. And a different number of (conductive) rubber strips and ridges may be provided on the detection device.

Although the embodiments above have been described in considerable detail, numerous variations and modifications will become apparent to those skilled in the art once the above disclosure is fully appreciated. It is intended that the following claims be interpreted to embrace all such variations and modifications.

The invention claimed is:

1. A detection device configured to be releasably attached to an absorbent article, comprising:
a first half provided with at least one strip of flexible conductive material; and
a second half;
wherein the absorbent article is provided with at least two conductive lines under its porous, liquid pervious layer, and
wherein when the detection device is attached to the absorbent article, its first and second halves are pinched towards each other under pressure, with the absorbent article being provided therebetween, and wherein the at least one strip of flexible conductive material under such pressure is pushed against the absorbent article and contacts the respective conductive lines through the porous, liquid pervious layer of the absorbent article.

2. A detection device configured to be releasably attached to an absorbent article, comprising:
a first half provided with at least two conducts; and
a second half provided with at least one strip of flexible material,
wherein the absorbent article is provided with at least two conductive lines under its porous, liquid pervious layer, and
wherein when the detection device is attached to the absorbent article, its first and second halves are pinched towards each other under pressure, with the absorbent article being provided therebetween, and wherein the at least one strip of flexible material under such pressure pushes the absorbent article against the first half, thereby making the at least two conductive lines in the absorbent article contact the respective contacts on the first half through the porous, liquid pervious layer of the absorbent article.

3. A detection device configured to be releasably attached to an absorbent article, comprising:
a first half, and
a second half;
wherein at least one strip of flexible material is provided on the inner side of one of the first and second halves, while at least one ridge is provided on the inner side of the other one of the first and second halves along the length direction of the detection device.
